(19) United States
(12) Patent Application Publication
(10) Pub. No.: US 2022/0251539 A1
(43) Pub. Date: Aug. 11, 2022

(54) CONCENTRATING BIOLOGICAL COMPONENTS

(71) Applicant: Hewlett-Packard Development Company, L.P., Spring, TX (US)

(72) Inventors: Si-Lam J. CHOY, Corvallis, OR (US); Hilary ELY, Corvallis, OR (US); John LAHMANN, Corvallis, OR (US); Uranbileg DAALKHAIJAV, Corvallis, OR (US); Sarah GISH, Corvallis, OR (US)

(73) Assignee: Hewlett-Packard Development Company, L.P., Spring, TX (US)

(21) Appl. No.: 17/630,613

(22) PCT Filed: Oct. 29, 2019
(86) PCT No.: PCT/US2019/058427

§ 371 Date: Jan. 27, 2022

(51) Int. Cl. C12N 15/10; B01L 3/00; G01N 1/40

(52) U.S. Cl. CPC C12N 15/1013; B01L 3/502761; B01L 2200/0631; B01L 2400/043; B01L 2200/0668; G01N 1/405

(57) ABSTRACT

A biological component concentration fluid assembly includes magnetizing microparticles that are surface-activated to bind with (or are bound to) a biological component; a multi-fluid density gradient column with a first fluid layer, a second fluid layer, and a third fluid layer; and a magnet to attract and draw the magnetizing microparticles from the first fluid layer, through the second fluid layer, and into the third fluid layer. The first fluid layer has a first fluid density, and a second fluid layer has a second fluid density that is greater than the first fluid density, and is positioned beneath the first fluid layer. A third fluid layer has a third fluid density that is greater than the second fluid density and is positioned beneath the second fluid layer. The second and third fluid layers in this example are formulated to interact with the surface of the magnetizing microparticles.

### BACKGROUND

In biomedical, chemical, and environmental testing, isolating a component of interest from a sample fluid can be useful. Such separations can permit analysis or amplification of a component of interest. As the quantity of available assays for components increases, so does the demand for the ability to isolate components of interest from sample fluids.

### BRIEF DESCRIPTION OF THE DRAWING

FIG. 1A graphically illustrates a schematic view of an example biological component concentration fluid assembly in accordance with examples of the present disclosure;

FIG. 1B graphically illustrates a schematic view of an example biological component concentration fluid assembly in accordance with examples of the present disclosure;

FIG. 2 is a flow diagram illustrating an example method of concentrating a biological component from a biological sample in accordance with examples of the present disclosure; and

FIG. 3 graphically illustrates an example of a microfluidic biological component concentration system in accordance with examples of the present disclosure.

### DETAILED DESCRIPTION

In biological assays, a biological component can be intermixed with other components in a biological sample that can interfere with subsequent analysis. As used herein, the term “biological component” can refer to materials of various types, including proteins, cells, cell nuclei, nucleic acids, bacteria, viruses, or the like, that can be present in a biological sample. A “biological sample” can refer to a fluid obtained for analysis from a living or deceased organism. Isolating the biological component from other components of the biological sample can permit subsequent analysis without interference and can increase an accuracy of the subsequent analysis. In addition, isolating a biological component from other components in a biological sample can permit analysis of the biological component that would not be possible if the biological component remained in the biological sample. Many of the current isolation techniques can include repeatedly dispersing and re-aggregating samples. The repeated dispersing and re-aggregating can result in a loss of a quantity of the biological component. Furthermore, isolating a biological component with some of these techniques can be complex, time consuming, and labor intensive and can also result in less than maximum yields of the isolated biological component.

In accordance with examples of the present disclosure, a biological component concentration fluid assembly includes magnetizing microparticles that are surface-activated to bind with a biological component, or which are bound to the biological component; a multi-fluid density gradient column with a first fluid layer, a second fluid layer, and a third fluid layer; and a magnet to attract and draw the magnetizing microparticles from the first fluid layer, through the second fluid layer, and into the third fluid layer. The multi-fluid density gradient column in this example includes a first fluid layer having a first fluid density, and a second fluid layer having a second fluid density that is greater than the first fluid density and positioned along the multi-fluid density gradient column beneath the first fluid layer. The second fluid layer in this example formulated to interact with a surface of the magnetizing microparticles when received from the first fluid layer of the multi-fluid density gradient column that is positioned thereabove. The multi-fluid density gradient column in this example also includes a third fluid layer having a third fluid density that is greater than the second fluid density and positioned along the multi-fluid density gradient column beneath the second fluid layer. The third fluid layer in this example is formulated to further interact with the surface of the magnetizing microparticles when received from the second fluid layer of the multi-fluid density gradient column that is positioned thereabove. In one example, the first fluid layer and the second fluid layer can be in direct fluid communication with one another and are phase separated from one another at a first fluid interface. Furthermore, the second fluid layer and the third fluid layer can be in direct fluid communication with one another and are phase separated from one another at a second fluid interface. The magnetizing microparticles can be loaded in the first fluid layer in one example. In another example, the magnetizing microparticles can be separate from the multi-fluid density gradient column and be formulated to be introduced to the first fluid layer. For example, the magnetizing microparticles can be dispersed in a loading fluid to be introduced to the first fluid layer to mix with the first fluid layer, or the loading fluid can form the first fluid layer with the pre-dispersed magnetizing microparticles. As an example, the first fluid layer can include a surface binding fluid where the biological component therein binds with a surface of the magnetizing microparticles, the second fluid layer can be a wash fluid, and the third fluid can be an elution fluid where the biological component is released from the surface of magnetizing microparticles. A density difference of the first fluid layer relative to the second fluid layer can be from about 50 mg/mL to about 3 g/mL. The magnetizing microparticles can include, for example, paramagnetic microparticles, superparamagnetic microparticles, dimagnetic microparticles, or a combination thereof. The magnet can be positioned below the multi-fluid density gradient column or positioned adjacent to a side of the multi-fluid density gradient column. The magnet can be positioned, movable, or positioned and movable to cause the magnetizing microparticles to downwardly move through the multi-fluid density gradient column.

In another example, microfluidic biological component concentration system includes magnetizing microparticles that are surface-activated to bind with a biological component, or which are bound to the biological component; a multi-fluid density gradient column with a first fluid layer and a second fluid layer; a magnet to attract and draw the magnetizing microparticles from the first fluid layer and into the second fluid layer; and a fluidic processing device fluidly coupled with the multi-fluid density gradient column to receive the biological component after passing through the multi-fluid gradient density column. The multi-fluid density gradient column in this example includes a first fluid layer having a first fluid density, and a second fluid layer having a second fluid density that is greater than the first fluid density and positioned along the multi-fluid density gradient column beneath the first fluid layer. The second fluid layer is formulated to interact with a surface of the magnetizing microparticles when received from the first fluid layer of the multi-fluid density gradient column that is positioned thereabove. In one specific example, a third fluid layer can be included having a third fluid density that is greater than the second fluid density and positioned along the multi-fluid density gradient column beneath the second fluid layer. The third fluid layer can be formulated to further interact with the surface of the magnetizing microparticles when received from the second fluid layer of the multi-fluid density gradient column that is positioned thereabove, wherein the fluidic processing device is fluidly coupled to the third fluid layer.

In another example, a method of concentrating a biological component from a biological sample includes loading a biological sample and magnetizing microparticles into a multi-fluid density gradient column. In this example, the biological sample includes a biological component and the magnetizing microparticles are surface-activated to become associated with or are pre-loaded with the biological component. The multi-fluid density gradient column in this example includes a first fluid layer having a first fluid density and which promotes a first interaction with a surface of the magnetizing microparticles, a second fluid layer having a second fluid density that is greater than the first fluid density and positioned along the multi-fluid density gradient column beneath the first fluid layer, and a third fluid layer having a third fluid density that is greater than the second fluid density and positioned along the multi-fluid density gradient column beneath the second fluid layer. The second fluid layer in this example is formulated to promote a second interaction with the surface when magnetizing microparticles are received therein from the first fluid layer, and the third fluid layer is formulated to promote a third interaction with the surface when magnetizing microparticles are received therein from the second fluid layer. The method also includes exposing the magnetizing microparticles to a magnetic field to move the magnetizing microparticles along with the biological component from the first fluid layer into the second fluid layer and from the second fluid layer into the third fluid layer. In one example, the method can include selectively withdrawing, e.g., pipetting, the biological component out of the third fluid layer. In one example, the biological component can be present in a cell, and the first fluid layer includes a lysing agent for the cell. In this example, the method can further include lysing cells in situ within the first fluid layer so that the biological component is liberated from the cell and binds with the magnetizing microparticles in the first fluid layer or after being magnetically moved into the second fluid layer. In another example, the magnetizing microparticles can be bound to the biological component in a loading fluid, and the loading fluid can be loaded onto the second fluid layer of the multi-fluid density gradient column to form the first fluid layer or to become an admixture with an already existing first fluid layer.

It is noted that when discussing a biological component concentration fluid assembly, a method of concentrating a biological component from a biological sample, or the microfluidic biological component concentration system herein, such discussions can be considered applicable to one another whether or not they are explicitly discussed in the context of that example. Thus, for example, when discussing a multi-fluid density gradient column in the biological component concentration fluid assembly, such disclosure is also relevant to and directly supported in the context of the method of concentrating a biological component from a biological sample, or the microfluidic biological component concentration system, and vice versa.

Terms used herein will have the ordinary meaning in the relevant technical field unless specified otherwise. In some instances, there are terms defined more specifically throughout the specification or included at the end of the present specification, and thus, these terms can have a meaning as described herein.

Biological Component Concentration Fluid Assemblies and Systems

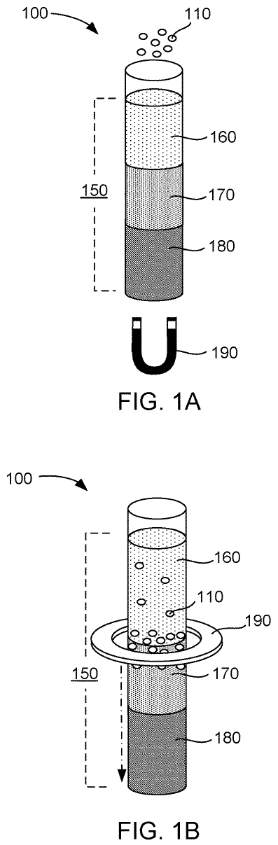

In accordance with examples of the present disclosure, a biological component concentration fluid assembly 100 is shown in FIG. 1A. The biological component concentration fluid assembly can include magnetizing microparticles 110, a multi-fluid density gradient column 150, and a magnet 190. The magnetizing microparticles can be surface-activated to bind with a biological component, or can be bound to the biological component. The multi-fluid density gradient column can include a first fluid layer 160, a second fluid layer 170, and a third fluid layer 180. The first fluid layer can have a first fluid density. In some examples, the first fluid layer can be formulated to interact with a surface of the magnetizing microparticles when introduced therein and/or can be a loading fluid that may or may not interact with the surface of the magnetizing microparticles. The second fluid layer can have a second fluid density that can be greater than the first fluid density and can be positioned along the multi-fluid density gradient column beneath the first fluid layer. The second fluid layer can be formulated to interact with the surface of the magnetizing microparticles when received from a fluid layer (e.g., either the first fluid layer or a fluid positioned between the first fluid layer and the second fluid layer) of the multi-fluid density gradient column that can be positioned thereabove. The third fluid layer can have a third fluid density that can be greater than the second fluid density and can be positioned along the multi-fluid density gradient column beneath the second fluid layer. The third fluid layer can be formulated to further interact with the surface of the magnetizing microparticles when received from a fluid layer of the multi-fluid density gradient column that is positioned thereabove. The magnet can be operable to attract and draw the magnetizing microparticles from the first fluid layer and into the second fluid layer.

In another example, as shown in FIG. 1B, a biological component concentration fluid assembly 100 can include magnetizing microparticles 110, a multi-fluid density gradient column 150, and a magnet 190. The magnetizing microparticles can be surface-activated to bind with a biological component, or can be bound to the biological component. The multi-fluid density gradient column can include a first fluid layer 160, a second fluid layer 170, and a third fluid layer 180, which can be similar to those described previously in FIG. 1A. However, as noted in FIG. 1B, the magnet 190 is a magnet positioned along a side of the multi-fluid density gradient column and may be being movable along a side of the column to move the magnetic particles vertically downward. Furthermore, this specific magnet is a ring magnet that can surround an exterior circumference of the multi-fluid density gradient column, though the movable magnet can be of any configuration or shape suitable for moving the magnetic particles vertically downward through the fluid layers of the multi-fluid density gradient column.

With regard to both FIGS. 1A and 1B, the first fluid layer 160 may be used for convenience in loading the magnetizing microparticles 110 into the multi-fluid density gradient column 150. For example, the first fluid layer in this instance may be preloaded with the magnetizing microparticles, and then that fluid can be loaded onto a top of the second fluid layer 170. The magnetizing microparticles are shown in this example as loaded within the first fluid layer, but as the magnet moves downward, most or many of magnetic particles transition out of the first fluid layer, across a fluid interface, and into the second fluid layer. As the magnet continues to move down, the magnetizing microparticles will, at a later point in the process, transition from within the second fluid layer, across a fluid interface, and into the third fluid layer 180.

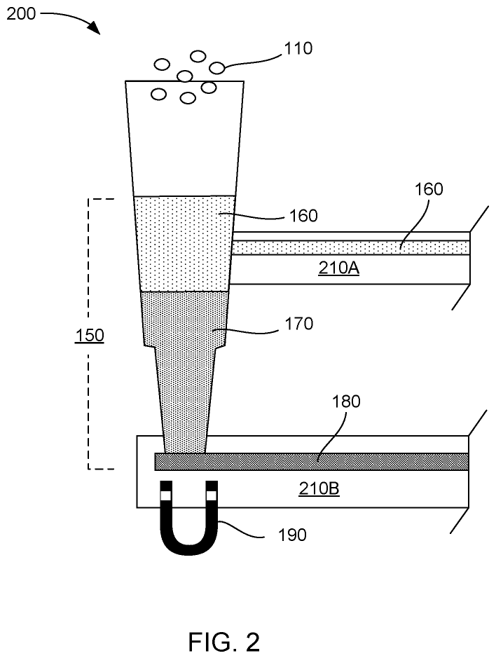

In a related example, a microfluidic biological component concentration system 200 is shown in FIG. 2. The system can include magnetizing microparticles 110; a multi-fluid density gradient column 150 including a first fluid layer 160, a second fluid layer 170, and a third fluid layer 180; and a magnet 190. The column in this example has a different geometry than that shown in FIGS. 1A and 1B, but is still arranged with vertical phase separated fluid layers. In this example, the magnet can attract and draw the magnetizing microparticles from the first fluid layer into the second fluid layer. The magnetizing microparticles can be surface-activated to bind with a biological component, or can be bound to the biological component. The first fluid layer can have a first fluid density, and in some examples, can be formulated to interact with a surface of the magnetizing microparticles when introduced therein along the multi-fluid gradient column, or can be a loading solution, etc. The second fluid layer can have a second fluid density that can be greater than the first fluid density and can be positioned along the multi-fluid density gradient column beneath the first fluid layer. The second fluid layer can be formulated to interact with the surface of the magnetizing microparticles when received from a fluid layer of the multi-fluid density gradient column that is positioned thereabove. The third fluid layer can have a third fluid density that can be greater than the second fluid density and positioned along the multi-fluid density gradient column beneath the second fluid layer. The third fluid layer can be formulated to further interact with the surface of the magnetizing microparticles when received from a fluid layer of the multi-fluid density gradient column that is positioned thereabove

In this specific example as shown in FIG. 2, the system 200 can also include a fluidic processing device 210A and/or 2106, which can be positioned downstream from the first fluid layer, but more typically downstream from the second fluid layer. Sometimes the fluidic processing device itself contains a fluid that is part of the multi-fluid density gradient column. As shown in this example, the first fluid layer 160 and/or the third fluid layer 180 of the multi-fluid density gradient column 150 can be partially or fully contained within the fluidic processing device(s). The fluidic processing device can be electromagnetically associated with the first fluid layer, the second fluid layer, or the third fluid layer along the multi-fluid density gradient column, or alternatively, the fluids thereof can be drawn from the first fluid layer, the second fluid layer, and/or the third fluid layer by other fluidic movement components, e.g., pumps, fluid ejectors, etc. In this example, as shown, fluidic processing device 210A is relative to the first fluid layer, e.g., prior to fluid introduction to the second fluid layer, and fluidic processing device 210B is relative to the third fluid layer (as well as the second fluid layer and the first fluid layer positioned thereabove). If a fluidic processing device(s) can be established to measure a property of a fluid that is fed to the fluidic processing device, this can occur prior to introduction of the magnetizing microparticles, while the magnetic particles are present in the fluid, after the magnetic particles have passed beyond the fluid into another fluid or location, or a combination thereof. Furthermore, the fluidic processing device(s) can receive a portion of the fluid for testing or assaying the fluid, for use of the fluid, for removal of a portion of or all of the fluid, etc. In one example, the fluidic processing device can be a microfluidic chip, such as a lab-on-a-chip device.

### Multi-Fluid Density Gradient Columns

The multi-fluid density gradient column can include a first fluid layer a second fluid layer, and a third fluid layer vertically arranged. A “multi-fluid density gradient column” as used herein, can refer to a multi-layered fluid column where individual fluid layers are separated from one another based on phase. A multi-fluid density gradient column does not include fluid layers where physical barriers separate one fluid layer from another. Fluid layers of the multi-fluid density gradient column can be phase separated from one another based on fluidic properties of the various fluids, including density of the respective fluids along the column. The greater or higher the density of a fluid, relative to other fluids in the column, the closer to the bottom of the column the fluid will be located. For example, the first fluid layer can have a first density and can form a first fluid layer of the multi-fluid density gradient column. The second fluid layer can have a second density that can be greater than a density of the first fluid layer and can form a second fluid layer of the multi-fluid density gradient column beneath the first fluid layer. The third fluid layer can have a third density that can be greater than a density of the second fluid layer and can form a third fluid layer of the multi-fluid density gradient column beneath the second fluid layer.

In some examples, a density of a fluid in a fluid layer can be altered using a densifier. Example densifiers can include sucrose, polysaccharides such as FICOLL™ (commercially available from Millipore Sigma (USA)), C₁₉H₂₆I₃N₃O₉ such as NYCODENZ® (commercially available from Progen Biotechnik GmbH (Germany)) or HISTODENZ™, iodixanols such as OPTIPREP™ (both commercially available from Millipore Sigma (USA)), or combinations thereof. In one example, a density difference of the first fluid layer relative to the second fluid layer can range from about 50 mg/mL to about 3 g/mL. In yet other examples, a density difference from the first fluid layer relative to the second fluid layer can range from about 50 mg/mL to about 500 mg/mL or from about 250 mg/mL to about 1 g/mL. In further detail, example additives that can be included in the first fluid layer, or in other fluid layers, depending on the design of the multi-fluid gradient column may include sucrose, heat eluted sucrose, C1-C4 alcohol, e.g., isopropyl alcohol, ethanol, etc., which can be included to adjust density, and/or to provide a function with respect to biological component or materials to pass through the column.

A quantity of fluid layers in the multi-fluid density gradient column is not particularly limited. In one example, the multi-fluid density gradient column can further include a fourth fluid layer having a fourth fluid density that can be greater than the third fluid density and can be positioned beneath the third fluid layer. The fourth fluid layer can be phase separated from the third fluid layer along a third fluid layer interface where the third fluid layer can be in fluid communication with the fourth fluid layer. In further examples, the assembly can further include a fifth, sixth, or seventh fluid layer that can be phase separated from the other fluids in the column based on a density of the fifth, sixth, or seventh fluid with respect to the other fluids in the column.

The fluid layers in the multi-fluid density gradient column can be formulated to interact with a surface of the magnetizing microparticles. Individual fluid layers can have a different function. For example, a fluid layer can include a lysis buffer to lyse cells. In yet other examples, a fluid layer can be a surface binding fluid layer to bind the biological component to the magnetizing microparticles, a wash fluid layer to trap contaminates from a sample fluid and/or remove contaminates from an exterior surface of the magnetizing microparticles, a surfactant fluid layer to coat the magnetizing microparticles, a dye fluid layer, an elution fluid layer to remove the biological component from the magnetizing microparticles following extraction from the biological sample, a labeling fluid layer for binding labels to the biological component such as a fluorescent label (either attached to the magnetizing microparticles or unbound thereto), a reagent fluid layer to prep a biological component for further analysis such as a master mix fluid layer to prep a biological component for PCR, and so on.

In some examples, individual fluid layers can provide sequential processing of a biological component from a biological sample. For example, individual fluid layers can carry out individual functions, and in many cases, the functions can be coordinated to achieve a specific result. Biological material that may be added can include whole blood, platelets, cells, lysed cells, cellular components, nucleic acids, e.g., DNA, RNA, primers, etc., oligo or poly-bases, peptides, or the like. More specifically, for example, in considering biological material found in a cell, sequential fluid layers from top to bottom of a multi-fluid density gradient column can act on the cell to lyse the cell in a first fluid layer, and bind a target biological material from the lysed cell to magnetic microparticles in a second fluid layer (or lysing and binding can alternatively be done in a single fluid). Additional fluid layers may be used to wash the magnetic microparticles with the biological material bound thereto in a third fluid layer, e.g., washing the second fluid layer from magnetic microparticles in the third fluid layer, and/or eluting (or separating) the biological material from the magnetic microparticles in the fourth fluid. The surface binding and cell lysis can occur, for example, with a lysate buffer in a sucrose and water solution. Washing can occur in a sucrose in water solution, for example. In other examples, one or more of the fluids can be present as a fluid layer(s) along the multi-fluid density gradient column in the form of a master mix fluid for nucleic acid processing. Other combinations of fluid layers (first, second, third, etc.) may include a surfacing binding fluid, a washing fluid, and an elution fluid; or may include a lysis fluid, a washing fluid, a surface binding fluid, a second washing fluid, an elution fluid, and a reagent fluid. Regardless of the various functions of the various fluid layers with sequentially increasing densities arranged from top to bottom, at the individual fluid layers, the magnetic microparticles can independently interact, e.g., become modified, with a fluid layer in order to sequentially process the magnetic microparticles with surface active groups and/or biological material associated therewith or associated with one or more of the fluid layers, for example.

A vertical height of the fluid layers in the multi-fluid density gradient column can vary. Adjusting a vertical height of a fluid layer can affect a residence time of the paramagnetic microparticles in that fluid layer. The taller the fluid layer, the longer the residence time of the magnetizing microparticles in the fluid layer. In some examples, all of the fluid layers in the multi-fluid density gradient column can be the same vertical height. In other examples, a vertical height of individual fluid layers in a multi-fluid density gradient column can vary from one fluid layer to the next. In one example, a vertical height of the first fluid layer and the second fluid layer along the multi-fluid density gradient column can individually range from about 10 μm to about 50 mm. In another example, a vertical height of the fluid layers along the multi-fluid density gradient column can individually range from about 10 μm to about 30 mm, from about 25 μm to about 1 mm, from about 200 μm to about 800 μm, or from about 1 mm to about 50 mm.

### Methods of Concentrating a Biological Components

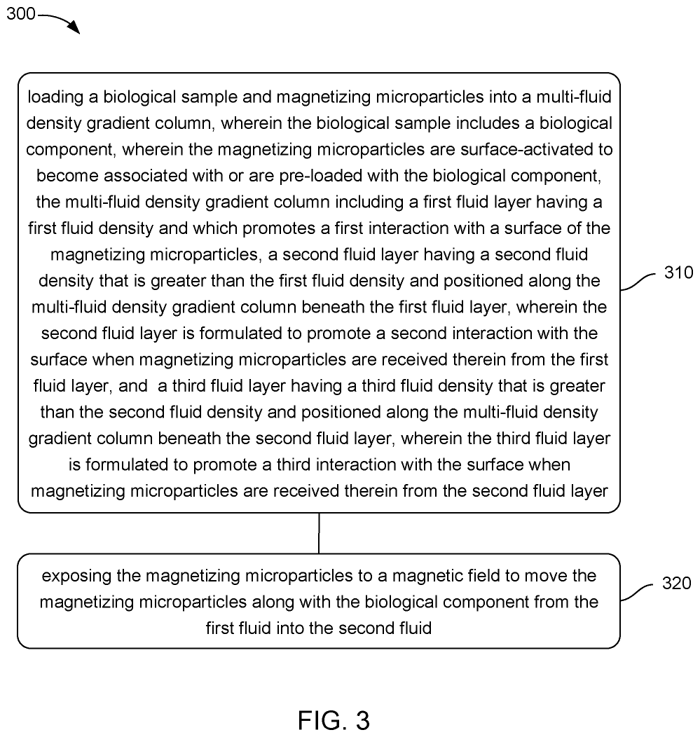

A flow diagram 300 of a method of concentrating a biological component from a biological sample is shown in FIG. 3. The method can include utilizing the biological component concentration fluid assembly described above, illustrated in FIG. 1 or 2, or other similar assemblies and/or systems. In this example, the method can include loading 310 a biological sample and magnetizing microparticles into a multi-fluid density gradient column. In this example, the biological sample includes a biological component and the magnetizing microparticles are surface-activated to become associated with or are pre-loaded with the biological component. The multi-fluid density gradient column in this example includes a first fluid layer having a first fluid density and which promotes a first interaction with a surface of the magnetizing microparticles, a second fluid layer having a second fluid density that is greater than the first fluid density and positioned along the multi-fluid density gradient column beneath the first fluid layer, and a third fluid layer having a third fluid density that is greater than the second fluid density and positioned along the multi-fluid density gradient column beneath the second fluid layer. The second fluid layer in this example is formulated to promote a second interaction with the surface when magnetizing microparticles are received therein from the first fluid layer, and the third fluid layer is formulated to promote a third interaction with the surface when magnetizing microparticles are received therein from the second fluid layer. The method also includes exposing 320 the magnetizing microparticles to a magnetic field to move the magnetizing microparticles along with the biological component from the first fluid layer into the second fluid layer and from the second fluid layer into the third fluid layer.

In some other examples, the biological sample including the biological component can be combined with the magnetizing microparticles in a loading solution prior to loading the biological sample including the biological component and the magnetizing microparticles into the multi-fluid density gradient column. For example, the magnetizing microparticles and the biological sample can be admixed in a loading fluid. The biological sample and the magnetizing microparticles can be permitted to incubate or otherwise become prepared for loading on top of or into the multi-fluid density gradient column. The magnetizing microparticles can bind with the biological component in the loading fluid and can then be added to the multi-fluid density gradient column for the fluid layers to act upon the magnetizing microparticles. In one example, the loading fluid can become the first fluid layer of the multi-fluid density gradient column. The second fluid layer, third fluid layer, (or any number of fluids present that are along the column and separated by the respective fluid densities) can further interact with a surface of the magnetizing micro particles.

The loading fluid (or the first fluid layer, or even the second fluid layer) can include secondary components selected from enzymes, cellular debris, lysing agents, buffers, or a combination thereof. The magnetizing microparticles can be bound to the biological component in a loading fluid or in a subsequent fluid along the multi-fluid density gradient column. In the case of a loading fluid, magnetizing microparticles including the biological component bound thereto can then be introduced as a separate fluid layer for the microparticles to be drawn into other fluid layers that can act on the microfluidic particles to further interact with the surface thereof along the multi-fluid density gradient column.

In one example, the method can further include selectively withdrawing, e.g., pipetting, the biological component out of the third fluid layer, such as through an ingress/egress opening(s) from the top, the bottom, or through a sidewall, for example. The biological component may still be associated with a surface of the magnetizing micro particles, or may be separated from the magnetizing microparticles. In another example, this method alternatively may include selectively withdrawing, e.g., pipetting, the first fluid layer, the second fluid layer, and/or the third fluid layer out of the multi-fluid density gradient column and leaving the magnetizing microparticles with the biological component bound thereto in a vessel of the multi-fluid density gradient column to either be further treated or removed after the extraction of the first fluid layer, the second fluid layer, and the third fluid layer therefrom. In some examples, the biological sample can include a cell and the biological component can be trapped within the cell. Lysing the cell can release the biological component therefrom and can permit isolation of the biological component. In this example, the first fluid layer or a loading fluid can include a lysing agent for the cell. The method can further include lysing the cell in situ within the first fluid layer or the loading fluid so that the biological component can be liberated from the cell and can bind with the magnetizing microparticles in the first fluid layer or the loading fluid.

### Magnetizing Microparticles

The magnetizing microparticles in the systems and methods describe herein can be in the form of paramagnetic microparticles, superparamagnetic microparticles, diamagnetic microparticles, or a combination thereof, for example. The magnetizing microparticles can likewise be surface-activated to bind with a biological component or can be bound to the biological component. The term “magnetizing microparticles” is defined herein to include microparticles that may not be magnetic in nature unless and until a magnetic field is introduced at a strength and proximity to cause them to become magnetic. Their magnetic strength can be dependent on the magnetic field applied and may get stronger as the magnetic flied is increased, or the magnetizing microparticles get closer to the magnetic source that is applying the magnetic field.

In more specific detail, “paramagnetic microparticles” have these properties, in that they have the ability to increase in magnetism when a magnetic field is present; however, paramagnetic microparticles are not magnetic when a magnetic field is not present. In some examples, the paramagnetic microparticles can exhibit no residual magnetism once the magnetic field is removed. A strength of magnetism of the paramagnetic microparticles can depend on the strength of the magnetic field, the distance between a source of the magnetic field and the paramagnetic microparticles, and a size of the paramagnetic microparticles. As a strength of the magnetic field increases and/or a size of the paramagnetic microparticles increases, the strength of the magnetism of the paramagnetic microparticles increases. As a distance between a source of the magnetic field and the paramagnetic microparticles increases the strength of the magnetism of the paramagnetic microparticles decreases. “Superparamagnetic microparticles” can act similar to paramagnetic microparticles; however, they can exhibit magnetic susceptibility to a greater extent than paramagnetic microparticles in that the time it takes to become magnetized appears to be near zero seconds. “Diamagnetic microparticles,” on the other hand, can display magnetism due to a change in the orbital motion of electrons in the presence of a magnetic field.

An exterior of the magnetizing microparticles can be surface-activated with surface groups that are interactive with a biological component of a biological sample or can include a covalently attached ligand attached to a surface of the microparticles to likewise bind with a biological component of a biological sample. In some examples, the ligand can include proteins, antibodies, antigens, nucleic acid primers, amino groups, carboxyl groups, epoxy groups, tosyl groups, sulphydryl groups, or the like. The ligand can be selected to correspond with and bind with the biological component and can vary based on the type of biological component being isolated from the biological sample. For example, the ligand can include a nucleic acid primer when isolating a biological component that includes a nucleic acid sequence. In another example, the ligand can include an antibody when isolating a biological component that includes antigen. Commercially available examples of magnetizing microparticles that are surface-activated include those sold under the trade name DYNABEADS®, available from ThermoFischer Scientific (USA).

The biological component concentration fluid assembly can include magnetizing microparticles, which can be, for example, paramagnetic microparticles, superparamagnetic microparticles, diamagnetic microparticles, or a combination thereof. Paramagnetic microparticles can have the ability to increase in magnetism when a magnetic field is present; however, paramagnetic microparticles are not magnetic when a magnetic field is not present. In some examples, the paramagnetic microparticles can exhibit no residual magnetism once the magnetic field is removed. A strength of magnetism of the paramagnetic microparticles can depend on the strength of the magnetic field, the distance between a source of the magnetic field and the paramagnetic microparticles, and a size of the paramagnetic microparticles. As a strength of the magnetic field increases and/or a size of the paramagnetic microparticles increases, a strength of the magnetism of the paramagnetic microparticles will be larger. As a distance between a source of the magnetic field and the paramagnetic microparticles increases, the strength of the magnetism of the paramagnetic microparticles decreases. Superparamagnetic microparticles can act similar to paramagnetic microparticles; however, they can exhibit magnetic susceptibility more quickly than paramagnetic microparticles in that the magnetization time appears to be near zero seconds. Diamagnetic microparticles can display magnetism due to a change in the orbital motion of electrons in the presence of a magnetic field.

An exterior of the magnetizing microparticles can be surface-activated with surface groups that are interactive with a biological component of a biological sample, or can include a covalently attached ligand attached to a surface of the microparticles to likewise bind with a biological component of a biological sample. In some examples, the ligand can include proteins, antibodies, antigens, nucleic acid primers, amino groups, carboxyl groups, epoxy groups, tosyl groups, sulphydryl groups, or the like. The ligand can be selected to correspond with and bind with the biological component and can vary based on the type of biological component being isolated from the biological sample. For example, the ligand can include a nucleic acid primer when isolating a biological component that includes a nucleic acid sequence. In another example, the ligand can include an antibody when isolating a biological component that includes antigen. Commercially available examples of magnetizing microparticles that are surface-activated include those sold under the trade name DYNABEADS® (available from ThermoFischer Scientific (USA)).

In some examples, the magnetizing microparticles can have an average particle size that can range from about 0.1 μm to about 70 μm. The term “average particle size” describes a diameter or average diameter, which may vary, depending upon the morphology of the individual particle. A shape of the magnetizing microparticles can be spherical, irregular spherical, rounded, semi-rounded, discoidal, angular, sub-angular, cubic, cylindrical, or any combination thereof. In one example, the particles can include spherical particles, irregular spherical particles, or rounded particles. The shape of the magnetizing microparticles can be spherical and uniform, which can be defined herein as spherical or near-spherical, e.g., having a sphericity of >0.84. Thus, any individual particles having a sphericity of <0.84 are considered non-spherical (irregularly shaped). The particle size of the substantially spherical particle may be provided by its diameter, and the particle size of a non-spherical particle may be provided by its average diameter (e.g., the average of multiple dimensions across the particle) or by an effective diameter, e.g., the diameter of a sphere with the same mass and density as the non-spherical particle. In further examples, the average particle size of the magnetizing microparticles can range from about 1 μm to about 50 μm, from about 5 μm to about 25 μm, from about 0.1 μm to about 30 μm, from about 40 μm to about 60 μm, or from about 25 μm to about 50 μm.

In an example, the magnetizing microparticles can be unbound to a biological component when added directly to a first fluid layer of a multi-fluid density gradient column. Binding between the magnetizing microparticles and the biological component of the biological sample can occur in the multi-fluid density gradient column. In yet another example, magnetizing microparticles and a biological sample including a biological component can be combined in a loading fluid before being added to a multi-fluid density gradient column. In this example, binding of the magnetizing microparticles to the biological component of the biological sample can occur in the multi-fluid density gradient column.

### Magnets

The biological component concentration fluid assembly can further include a magnet that can be capable of generating a magnetic field, such as a magnetic field that can be turned on and off by introducing electrical current/voltage to the magnet. Alternatively, the magnet can be a permanent magnet that is placed in proximity to the multi-fluid density gradient column to effect the movement of the magnetizing microparticles. The magnet can be permanently placed within this proximity, or can be movable along the column, or movable in position and/or out of position to effect movement of the magnetizing microparticles. The magnetizing microparticles can be magnetized by the magnetic field generated by the magnet. In addition, the magnet can create a force capable of pulling the magnetizing microparticles through the multi-fluid density gradient column. When the magnet is turned off or not in appropriate proximity, the magnetizing microparticles can reside in a fluid layer until gravity pulls the magnetizing microparticles through fluid layers of the multi-fluid density gradient column, or they may remain suspended in the fluid layer in which they may reside until the magnetic field is applied thereto. The rate at which gravity pulls the magnetizing microparticles through fluid layers (or leaves the magnetizing microparticles within a fluid layer) can be based on a mass of the magnetizing microparticles in combination with a surface tension between fluid layers. The magnet can cause the magnetizing microparticles to move from one fluid layer to another, or increase a rate at which the magnetizing microparticles pass from one fluid layer into another.

In an example, the magnet can be positioned below the multi-fluid density gradient column, as illustrated in FIGS. 1A and 2, and can be in a fixed position or can be moveable in position, out of position, or at variable positions to effect downward movement, rate of movement, or to promote little to no movement of the magnetizing microparticles. In another example, the magnet can be positioned adjacent to a side of the multi-fluid density gradient column and can move vertically to cause the magnetizing microparticles to move therewith. In some examples, the magnet can be a ring magnet, as shown in FIG. 1B. A movable magnet(s) can likewise be positioned adjacent to a side of the multi-fluid density gradient column that is not a ring shape, but can be any shape effective for moving magnetizing microparticles along the column. In some examples, the magnet can be moved along a side and/or along a bottom of the multi-fluid density gradient column to pull the magnetizing microparticles in one direction or another. In one example, the magnet can be used to pull the magnetizing microparticles downwardly through fluid layers of the multi-fluid density gradient column. In yet other examples, the magnet can be used to concentrate the magnetizing microparticles near a side wall of the multi-fluid density gradient column to be moved downward by a movable magnet, or by a magnet positioned beneath the multi-fluid density gradient column. In one example, a magnet used to move magnetizing microparticles downward can be used to reverse the direction of the magnetizing microparticles and can cause the magnetizing microparticles to re-enter a fluid layer that the magnetizing microparticles have previously passed through.

A strength of the magnetic field and the location of the magnet in relation to the magnetizing microparticles can affect a rate at which the magnetizing microparticles move downwardly through the multi-fluid density gradient column. The further away the magnet and the lower the strength of the magnetic field, the slower the magnetizing microparticles will pass through the multi-fluid density gradient column. In an example, a maximum distance between the magnet and a nearest location where the first fluid layer resides along the multi-fluid density gradient column can be about 50 mm, about 40 mm maximum distance, about 30 mm maximum distance, about 20 mm maximum distance, or about 10 mm maximum distance. The minimum distance, on the other hand, may be from about 0.1 mm minimum distance, from about 1 mm minimum distance, or about 5 mm minimum distance. In one example, the minimum distance between the magnet and the multi-fluid density gradient column may be about the thickness of the container or vessel that contains the multi-fluid density gradient column. Thus, distance ranges between the magnet and the multi-fluid density gradient column can be from about 0.1 mm to about 50 mm, from about 1 mm to about 50 mm, from about 1 about mm to about 40 mm, from about 1 mm to about 30 mm, from about 1 mm to about 20 mm, from about 1 mm to about 10 mm, from about 5 mm to about 50 mm, or from about 5 mm to about 30 mm. In another example, a maximum distance between the magnet and a nearest location where the first fluid layer resides along the multi-fluid density gradient column can be about 30 mm.

### Definitions

It is noted that, as used in this specification and the appended claims, the singular forms “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and determined based on experience and the associated description herein.

As used herein, the phrase “in fluid communication” indicates that two or more fluids are fluidly coupled to one another, either directly or in some instances with intervening fluid(s) therebetween. In accordance with this definition, the term “in fluid communication” excludes fluids that are separate by physical barrier, but rather are phase separated by density, for example.

As used herein, the term “interact” or “interaction” as it relates to a surface of the magnetizing microparticles indicates that a chemical, physical, or electrical interaction occurs where a magnetizing microparticle surface property is modified in some manner that are different than may have been present prior to entering the fluid layer, but does not include modification of magnetic properties magnetizing microparticles as they are influenced by the magnetic field introduced by the magnet. For example, a fluid layer can include a lysis buffer to lyse cells, and cellular components can become associated with a surface of the magnetizing microparticles. Lysing cells in a fluid can modify the fluid sample and thus modify or interact with a surface of magnetizing microparticles, e.g., the cellular component binds or becomes associated with a surface of the magnetizing microparticles. In yet other examples, a fluid layer that would be considered to interact with the magnetizing microparticles could be a wash fluid layer to trap contaminates from a sample fluid and/or remove contaminates from an exterior surface of the magnetizing microparticles, a surfactant fluid layer to coat the magnetizing microparticles, a dye fluid layer to introduce visible or other markers to the fluid or surface, an elution fluid layer to remove the biological component from the magnetizing microparticles following extraction from the biological sample, a labeling fluid layer for binding labels to the biological component such as a fluorescent label (either attached to the magnetizing microparticles or unbound thereto), a reagent fluid layer to prep a biological component for further analysis such as a master mix fluid layer to prep a biological component for PCR, and so on.

As used herein, a plurality of items, structural elements, compositional elements, and/or materials may be presented in a common list for convenience. However, these lists should be construed as though individual members of the list is individually identified as a separate and unique member. Thus, no individual member of such list should be construed as a de facto equivalent of any other member of the same list solely based on presentation in a common group without indications to the contrary.

Concentrations, amounts, and other numerical data may be expressed or presented herein in a range format. A range format is used merely for convenience and brevity and thus should be interpreted flexibly to include not only the numerical values explicitly recited as the limits of the range, but also to include individual numerical values or sub-ranges encompassed within that range as if numerical values and sub-ranges are explicitly recited. As an illustration, a numerical range of “about 1 wt % to about 5 wt %” should be interpreted to include not only the explicitly recited values of about 1 wt % to about 5 wt %, but also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting one numerical value. Furthermore, such an interpretation should apply regardless of the breadth of the range or the characteristics being described.

### Examples

The following illustrates several examples of the present disclosure. However, the following are illustrative of the application of the principles of the present disclosure. Numerous modifications and alternative compositions, methods, and systems may be devised without departing from the spirit and scope of the present disclosure. The appended claims are intended to cover such modifications and arrangements.

### Example 1—Multi-Fluid Density Gradient Column Separation

DNA was extracted from a 2.5×10⁵ live Streptococcus thermophilus bacteria using a multi-fluid density gradient column in accordance with the present disclosure. Several different multi-fluid density gradient columns were prepared in 1.7 mL micro-centrifuge tubes. The top fluid layer (first fluid layer) included 300 μg DYNABEADS® DNA Direct Universal paramagnetic microparticles in 100 μL lysis buffer (from the Dynabeads DNA Direct Universal kit), which are commercially available from ThermoFisher Scientific (USA). The intermediate fluid layer (second fluid layer) of the multi-fluid density gradient columns included 0.25 g/mL sucrose in 50 vol % ethanol and water to provide a washing layer. The lowest fluid layer (third fluid layer) of the multi-fluid density gradient column included 1 g/mL sucrose in ultrapure H₂O with blue dye added thereto.

The live Streptococcus thermophilus bacteria was added to the first fluid layer and allowed to incubate for 2 minutes during which the cells were chemically lysed and the extracted genomic DNA bound to the Dynabeads. Following the incubation period, a permanent rare earth magnet with 1 cm² surface area was placed beneath the multi-fluid gradient column and the magnetizing microparticles with DNA attached or attracted to the surfaces thereof were passed from the respective first fluid layer into the second fluid layer and the third fluid layer. After the fluids had had time to act on and respectively interact with the surfaces of the magnetic microparticles, the first fluid layer, the second fluid layer, and the third fluid layer were pipetted off from the multi-fluid density gradient column, leaving the magnetizing microparticles in the bottom of the micro-centrifuge tubes.

The magnetizing microparticles with the DNA bound thereto were re-suspended in 10 μL of master mix containing DNA polymerases, magnesium, dNTPS, primers, hydrolysis probes, bovine serum albumin, and buffer solution, and transferred to a PCR reaction vessel. PCR was carried out using Bio-Rad CFX96 Touch Real-Time PCR thermocycler. The passing of the magnetizing microparticles through the multi-fluid density gradient column did not significantly affect the PCR reaction times.

### Example 2—Multi-Fluid Density Gradient Column Separation

DNA was extracted from a 2.5×10⁵ live Streptococcus thermophilus bacteria in triplicate using a biological component concentration fluid assembly. A multi-fluid density gradient column with three fluid layers was formed in a 1.7 mL micro-centrifuge tube. The top fluid layer was as described below. The intermediate fluid layer included 200 μL 500 mg/mL sucrose solution with 1 μL red food dye for ease of observation. The lowest fluid layer included 200 μL protein blocking agent in 1 g/mL sucrose solution.

The Streptococcus thermophilus bacteria was admixed in a top fluid layer (or pre-mixed in a fluid and added to the column as a top layer), which included 200 μL lysis buffer fluid with 300 μg DYNABEADS® DNA Direct Universal magnetizing microparticles, commercially available from ThermoFisher Scientific (USA). The first fluid used to form the first fluid layer was allowed to incubate for 2.5 minutes and added over the intermediate fluid layer to form the top fluid layer of the multi-fluid density gradient column. Following the incubation period, a ring magnet shielded on one side was placed beneath the multi-fluid gradient column and the DYNABEADS® DNA Direct Universal magnetizing microparticles were passed from the top fluid layer (first fluid layer), through the intermediate fluid layer (second fluid layer), and into the lower fluid layer (third fluid layer). Passing of the magnetizing microparticles with the DNA bound thereto from fluid layer to fluid layer in a downward direction due to the application of the magnetic field thereto did not impact the biological material for subsequent processing.

While the present technology has been described with reference to certain examples, various modifications, changes, omissions, and substitutions can be made without departing from the spirit of the disclosure. The disclosure be limited only by the scope of the following claims.

What is claimed is:

1. A biological component concentration fluid assembly, comprising
magnetizing microparticles that are surface-activated to bind with a biological component, or which are bound to the biological component;
a multi-fluid density gradient column, including:
a first fluid layer having a first fluid density, and
a second fluid layer having a second fluid density that is greater than the first fluid density and positioned along the multi-fluid density gradient column beneath the first fluid layer, wherein the second fluid layer is formulated to interact with a surface of the magnetizing microparticles when received from the first fluid layer of the multi-fluid density gradient column that is positioned thereabove;
a third fluid layer having a third fluid density that is greater than the second fluid density and positioned along the multi-fluid density gradient column beneath the second fluid layer, wherein the third fluid layer is formulated to further interact with the surface of the magnetizing microparticles when received from the second fluid layer of the multi-fluid density gradient column that is positioned thereabove; and
a magnet to attract and draw the magnetizing microparticles from the first fluid layer, through the second fluid layer, and into the third fluid layer.

2. The biological component concentration fluid assembly of claim 1, wherein the first fluid layer and the second fluid layer are in direct fluid communication with one another and are phase separated from one another at a first fluid interface, and wherein the second fluid layer and the third fluid layer are in direct fluid communication with one another and are phase separated from one another at a second fluid interface.

3. The biological component concentration fluid assembly of claim 1, wherein the magnetizing microparticles are loaded in the first fluid layer.

4. The biological component concentration fluid assembly of claim 1, wherein the magnetizing microparticles are separate from the multi-fluid density gradient column and are formulated to be introduced to the first fluid layer.

5. The biological component concentration fluid assembly of claim 1, wherein the magnetizing microparticles are dispersed in a loading fluid to be introduced to the first fluid layer to mix with the first fluid layer, or the loading fluid becomes the first fluid layer with the pre-dispersed magnetizing microparticles.

6. The biological component concentration fluid assembly of claim 1, wherein the first fluid layer includes a surface binding fluid where the biological component therein binds with a surface of the magnetizing microparticles, the second fluid layer is a wash fluid, and the third fluid is an elution fluid where the biological component is released from the surface of magnetizing microparticles.

7. The biological component concentration fluid assembly of claim 1, wherein a density difference of the first fluid layer relative to the second fluid layer is from about 50 mg/mL to about 3 g/mL.

8. The biological component concentration fluid assembly of claim 1, wherein the magnetizing microparticles include paramagnetic microparticles, superparamagnetic microparticles, dimagnetic microparticles, or a combination thereof.

9. The biological component concentration fluid assembly of claim 1, wherein the magnet is positioned below the multi-fluid density gradient column, or positioned adjacent to a side of the multi-fluid density gradient column, wherein the magnet is positioned, movable, or positioned and movable to cause the magnetizing microparticles to downwardly move through the multi-fluid density gradient column.

10. A microfluidic biological component concentration system, comprising
magnetizing microparticles that are surface-activated to bind with a biological component, or which are bound to the biological component;
a multi-fluid density gradient column, including:
a first fluid layer having a first fluid density, and
a second fluid layer having a second fluid density that is greater than the first fluid density and positioned along the multi-fluid density gradient column beneath the first fluid layer, wherein the second fluid layer is formulated to interact with a surface of the magnetizing microparticles when received from the first fluid layer of the multi-fluid density gradient column that is positioned thereabove;
a magnet to attract and draw the magnetizing microparticles from the first fluid layer and into the second fluid layer; and
a fluidic processing device fluidly coupled with the multi-fluid density gradient column to receive the biological component after passing through the multi-fluid gradient density column.

11. The system of claim 10, further comprising a third fluid layer having a third fluid density that is greater than the second fluid density and positioned along the multi-fluid density gradient column beneath the second fluid layer, wherein the third fluid layer is formulated to further interact with the surface of the magnetizing microparticles when received from the second fluid layer of the multi-fluid density gradient column that is positioned thereabove, wherein the fluidic processing device is fluidly coupled to the third fluid layer.

12. A method of concentrating a biological component from a biological sample, comprising:
loading a biological sample and magnetizing microparticles into a multi-fluid density gradient column, wherein the biological sample includes a biological component, wherein the magnetizing microparticles are surface-activated to become associated with or are pre-loaded with the biological component, the multi-fluid density gradient column including:
a first fluid layer having a first fluid density and which promotes a first interaction with a surface of the magnetizing microparticles,
a second fluid layer having a second fluid density that is greater than the first fluid density and positioned along the multi-fluid density gradient column beneath the first fluid layer, wherein the second fluid layer is formulated to promote a second interaction with the surface when magnetizing microparticles are received therein from the first fluid layer, and
a third fluid layer having a third fluid density that is greater than the second fluid density and positioned along the multi-fluid density gradient column beneath the second fluid layer, wherein the third fluid layer is formulated to promote a third interaction with the surface when magnetizing microparticles are received therein from the second fluid layer; and
exposing the magnetizing microparticles to a magnetic field to move the magnetizing microparticles along with the biological component from the first fluid layer into the second fluid layer and from the second fluid layer into the third fluid layer.

13. The method of claim 12, further comprising selectively withdrawing the biological component out of the third fluid layer.

14. The method of claim 12, wherein the biological component is present in a cell, and the first fluid layer includes a lysing agent for the cell, and the method includes lysing cells in situ within the first fluid layer so that the biological component is liberated from the cell and binds with the magnetizing microparticles in the first fluid layer or after being magnetically moved into the second fluid layer.

15. The method of claim 12, wherein the magnetizing microparticles are bound to the biological component in a loading fluid, and the loading fluid is loaded onto the second fluid layer of the multi-fluid density gradient column to form the first fluid layer or is loaded into the first fluid layer to become an admixture with the first fluid layer.
